SEQUENCE LISTING (12 sequences)

SEQ ID NO: 1 - protein, 35 aa, Artificial Sequence
  Synthetic Peptide
AKKYAKKEKAAKKAYKKEAKAKAAEAAAKEAAYEA

SEQ ID NO: 2 - protein, 45 aa, Artificial Sequence
  Synthetic Peptide
AKKYAKKAKAEKAKKAYKAAEAKKAAKYEKAAAEKAAAKEAAYEA

SEQ ID NO: 3 - protein, 56 aa, Artificial Sequence
  Synthetic Peptide
AKKYAKKEKAYAKKAEKAAKKAEAKAYKAAEAKKKAEAKYKAEAAKAAAKEAAYEA

SEQ ID NO: 4 - protein, 66 aa, Artificial Sequence
  Synthetic Peptide
AKKYAKKEKAYAKAKKAEAKAAKKAKAEAKKYAKAAKAEKKEYAAAEAKYKAEAAKAAAKEAAYEA

SEQ ID NO: 5 - protein, 77 aa, Artificial Sequence
  Synthetic Peptide
AKKYAKKEKAYAKKAEKAAKKAEAKAYKAAEAKKKAKAEAKKYAKAAKAEKKEYAAAEAKYKAEAAKAAAKEAAYEA

SEQ ID NO: 6 - protein, 86 aa, Artificial Sequence
  Synthetic Peptide
AKKYAKKEKAYAKKAEKAAKKAEAKAYKAAEAKKKAKAEAKKYAKAAKAEKKEYAAAEAKYKAEAAKKAYKAEAAKAAAKEAAYEA

SEQ ID NO: 7 - protein, 109 aa, Artificial Sequence
  Synthetic Peptide
AKKYAKKAEKAYAKKAKAAKEKKAYAKKEAKAYKAAEAKKKAKAEAKKYAKEAAKAKKEAYKAEAKKYAKAAKAEKKEYAAAEAKKAEAAKAYKAEAAKAAAKEAAYEA

SEQ ID NO: 8 - protein, 149 aa, Artificial Sequence
  Synthetic Peptide
AEKYKAKKAKEKAYKKKAKEAKKAKYKAKEAKAYKAEKKAKYAKAKEKAYAKAKEAKAYAKAKAKAEKAKAKAKYAEKAKAAKYAEKAAKYAEAKAKAAEAKYAAEAKEAAKAAEAKYAAKAEAAKYAAEKAAEKYAKAEAAAEAKEAA

SEQ ID NO: 9 - protein, 169 aa, Artificial Sequence
  Synthetic Peptide
AKKKYKAKEKKAKKKAKEKKYKAKKAKYKEKAAKYKAKKAKAKYKAKAEKAKAKAEKAKAYAEKAKAKYAKEAKKYAEKAKKAEYKAKEAAEKAKAYAKEAAKAEKAAKAAEKAAKAYAKAEAAAKEAAYAAKAEAKAAYAAEAAKAEYAAEAAKEAAYAAAEYAAEAA

SEQ ID NO: 10 - protein, 190 aa, Artificial Sequence
  Synthetic Peptide
AKKKYKAKEKKAKKKAKEKKKYKAKEKKAKKYKEKAAKYKAKKAKKEAAKYKKAKAEKAKYAKEKAEKAKAYAEKAKAKYAKEAKKYAEKAKKAEYKAKEAAEKAKAYAKEAAKAEAKAAKYAAEKAAEAAKAAYAKAEAAKAAKEAAYAAKAEAAKAAYAAEAAKAEYAAEAAKEAAYAAAEYAAAEAA

SEQ ID NO: 11 - protein, 221 aa, Artificial Sequence
  Synthetic Peptide
AKKKYKAKEKKAKAKKKAKEKKKYKAKKEKKAKKYKEKAAKYKAKKEKAKKEAAKYKKAKKYKAEKAKYAKEKAKEKAKAYAEKAEKAAKYAAKEAKKYAEKAAEKKAEYKAKEAAEKYAAKAYAAKEAAKAYKEAKAAKYAKAAEKAAAKEAAKAAYAAKAEAAKEAAKEAAYAAAKAEAAAAKAAAYAAEAAAKAEYAAEAAAKEAAYAAAEYAAAEAA

SEQ ID NO: 12 - protein, 249 aa, Artificial Sequence
  Synthetic Peptide
AKKKYKAKEKKAKYKAKKEKKAKEKKAKKKYKAKKKAAEKKYAKEKKAKEKAAKKYKAKKEKAKKEAAKYKKAKKYKAEKKAKYAAKEKAKEKAKAYAEKKAEKAAKYAAKEAKKYAEKAAEKKAEYKAKEAAEKYAAKKAEYAAEKEAAKAYKEAKAAKYAKAAEKAKAAKEAAKAAYAAKAEAAKEAAKEAAYAAAKAEAAAYAAAKAAAAYAAEAAAKAEYAAAEAAAKEAAYAAAEYAAAAEAAA

(19) United States
(12) Patent Application Publication
(10) Pub. No.: US 2008/0118553 A1
(43) Pub. Date: May 22, 2008

(54) Tannate salt form of polypeptide mixtures, their preparation and use

(76) Inventors: Anton Frenkel, Modiim (IL); Arthur A. Komlosh, Herzliya (IL)

Correspondence Address: COOPER & DUNHAM, LLP, 1185 AVENUE OF THE AMERICAS, NEW YORK, NY, 10036, US

(21) Appl. No.: 11/811,684

(22) Filed: Jun. 12, 2007

Related U.S. Application Data
(60) Provisional application No. 60/813,303, filed on Jun. 12, 2006.

(51) Int. Cl. A61K 38/17; A61K 48/00; A61K 31/70; A61K 39/395; A61K 31/55; A61K 31/5415; A61K 31/522; A61K 31/375; A61K 31/485; A61K 38/22; A61K 39/08

(52) U.S. Cl. 424/451; 514/8; 977/906; 424/489; 424/470; 514/291; 514/305; 514/53; 514/44; 514/183; 514/224.8; 514/665; 514/389; 514/304; 424/145.1; 514/263.31; 424/239.1; 514/559; 514/282; 514/725; 514/474; 514/458; 424/752; 514/217

(57) ABSTRACT

The subject invention provides a composition comprising a mixture of polypeptides in the form of a tannate salt wherein each polypeptide is a copolymer of the amino acids L-glutamic acid, L-alanine, L-tyrosine and L-Lysine, methods of preparation and uses thereof.

This application claims the benefit of U.S. Provisional Application No. 60/813,303, filed Jun. 12, 2006, the contents of which are hereby incorporated by reference.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Copolymers of L-glutamic acid, L-alanine, L-tyrosine, and L-lysine and mixtures thereof have long been known. (e.g., U.S. Pat. No. 3,849,550, issued Nov. 19, 1974 to Teitelbaum, et al., and U.S. Pat. No. 5,800,808, issued Sep. 1, 1998 to Konfino et al.) Over the last two decades such copolymer mixtures have been extensively studied, and numerous modifications as well as potential uses have been identified. These efforts have led to a commercial product, COPAXONE®, a therapeutic agent to treat multiple sclerosis (MS). (Physician's Desk Reference, 2005, Medical Economics Co. Inc., Montvale, N.J., 3115))

COPAXONE® is the brand name for a pharmaceutical composition which contains glatiramer acetate (GA) as the active ingredient. COPAXONE® contains the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. Chemically, glatiramer acetate is designated L-glutamic acid polymer with L-alanine, L-lysine and L-tyrosine, acetate (salt) Its structural formula is:

(Glu, Ala, Lys, Tyr)ₓ._{X}CH₃COOH (C₅H₉NO₄.C₃H₇NO₂.C₆H₁₄N₂O₂.C₉H₁₁NO₃)ₓ._{X}C₂H₄O₂

### CAS—147245-92-9

(“Copaxone”, Physician's Desk Reference, (2005), Medical Economics Co., Inc., (Montvale, N.J.), 3115.)

More recently, a new polypeptide mixture with a molecular weight range higher than that of glatiramer acetate has been developed (PCT International Application Publication No. WO 2006/029411).

Glatiramer acetate is approved for use in the reduction of the frequency of relapses in patients with relapsing-remitting multiple sclerosis. Multiple sclerosis has been classified as an autoimmune disease. Glatiramer acetate has also been disclosed for use in the treatment of other autoimmune diseases (Publication No. US 2002/0055466 A1, published May 9, 2002, (R. Aharoni et al.)), inflammatory non-autoimmune diseases (Publication No. US 2005/0014694 A1, published Jan. 20, 2005 (V. Wee Yong et al.)); and U.S. Patent Application No. 2002/0077278 A1, published June 20, 2002 (Young et al.).) and to promote nerve regeneration and/or to prevent or inhibit secondary degeneration which may follow primary nervous system injury (Publication No. US 2003/0004099 A1, published Jan. 8, 2004) (M. Eisenbach-Schwartz et al.)); and U.S. Patent Publication No. 2002/0037848 A1, published Mar. 28, 2002 (Eisenbach-Schwartz)). Furthermore, glatiramer acetate has been disclosed as a treatment for immune mediated diseases (e.g., U.S. Pat. No. 6,514,938 B1, issued Feb. 4, 2003 (Gad et al.); PCT International Publication No. WO 01/60392, published Aug. 23, 2001 (Gilbert et al.); and PCT International Publication No. WO 00/27417, published May 19, 2000 (Aharoni et al.)) as well as diseases associated with demyelination (PCT International Publication No. WO 01/97846, published Dec. 27, 2001 (Moses et al.)).

COPAXONE® is an injectable form of glatiramer acetate for the treatment of multiple sclerosis. Other approved treatments of multiple sclerosis also involve subcutaneous injection. Drawbacks of injection-based treatments include frequently observed injection-site reactions such as irritation, hypersensitivity, inflammation and pain, along with a reduced patient compliance. However, despite its desirability, an oral form of polypeptide mixture drugs such as glatiramer acetate has been elusive. Significant efforts to develop oral glatiramer acetate, including clinical testing, have thus far resulted in failure (Filippi et al., Lancet Neurol. 2006; 5:213-220)

This invention provides mixtures of polypeptides which are suitable for oral administration.

### SUMMARY OF THE INVENTION

The subject invention provides a composition comprising a mixture of polypeptides in the form of a tannate salt wherein each polypeptide consists of the amino acids L-glutamic acid, L-alanine, L-tyrosine and L-Lysine, and wherein the polypeptides in the mixture do not all have the same amino acid sequence.

The subject invention also provides a pharmaceutical composition comprising a therapeutically effective amount of the composition described herein and a pharmaceutically acceptable carrier.

The subject invention also provides a process for making a mixture of tannate salt of polypeptides, wherein each polypeptide consists of the amino acids L-glutamic acid, L-alanine, L-tyrosine and L-lysine, and wherein the polypeptides in the mixture do not all have the same amino acid sequence, comprising:
a) obtaining a mixture of acetate salt of polypeptides, wherein each polypeptide consists of the amino acids L-glutamic acid, L-alanine, L-tyrosine and L-lysine, and wherein the polypeptides in the mixture do not all have the same amino acid sequence; and
b) contacting the mixture of acetate salt of the polypeptides of step a) with tannic acid under suitable conditions to thereby form the mixture of tannate salt of the polypeptide.

The subject invention also provides a method of treating a human subject afflicted with an autoimmune disease comprising administering to the subject a therapeutically effective amount of the composition described herein, or of the pharmaceutical composition described herein, so as to treat the human subject.

The subject invention also provides a method of treating a human subject afflicted with an inflammatory non-autoimmune disease, an immune mediated disease, or a disease associated with demyelination comprising administering to the human subject a therapeutically effective amount of the composition described herein, or of the pharmaceutical composition described herein, so as to treat the human subject.

The subject invention also provides a method of alleviating a symptom of an autoimmune disease in a subject afflicted with such a disease, comprising administering to the human subject the composition described herein, or of the pharmaceutical composition described herein in an amount effective to alleviate the symptom.

The subject invention also provides a method of alleviating a symptom of an inflammatory non-autoimmune disease, an immune mediated disease, or a disease associated with demyelination in a subject afflicted with such a disease, comprising administering to the human subject the composition described herein, or of the pharmaceutical composition described herein in an amount effective to alleviate the symptoms.

The subject invention also provides a method of promoting nerve regeneration or preventing or inhibiting secondary degeneration which may otherwise follow primary nervous system injury in a human subject comprising administering to the human subject a therapeutically effective amount of the composition described herein, or of the pharmaceutical composition described herein.

The subject invention also provides a method of treating a human subject afflicted with a neurodegenerative disease comprising administering to the human subject a therapeutically effective amount of the composition described herein, or of the pharmaceutical composition described herein so as to thereby treat the human subject.

The subject invention also provides a method of alleviating a symptom of an neurodegenerative disease comprising administering to the human subject the composition described herein, or of the pharmaceutical composition described herein in an amount effective to alleviate the symptom.

The subject invention also provides a method of treating a human subject afflicted with an inflammatory bowel disease comprising administering to the human subject a therapeutically effective amount of the composition described herein, or of the pharmaceutical composition described herein so as to treat of the inflammatory bowel disease.

The subject invention also provides a method of alleviating a symptom of an inflammatory bowel disease comprising administering to the human subject the composition described herein, or of the pharmaceutical composition described herein in an amount effective to alleviate the symptom.

The subject invention also provides a method of treating a human subject afflicted with multiple sclerosis comprising administering to the human subject a therapeutically effective amount of the composition described herein, or of the pharmaceutical composition described herein so as to thereby treat the human subject afflicted with multiple sclerosis.

The subject invention also provides a method of alleviating a symptom of multiple sclerosis in a human subject afflicted with multiple sclerosis comprising administering to the human subject the composition described herein, or of the pharmaceutical composition described herein in an amount effective to alleviate the symptom of multiple sclerosis.

The subject invention also provides a method of reducing the frequency of relapses in a human subject afflicted with relapse remitting multiple sclerosis comprising administering to the human subject a therapeutically effective amount of the composition described herein, or of the pharmaceutical composition described herein so as to thereby reduce the frequency of relapses in the human subject.

The subject invention also provides a method of reducing the disability based on the EDSS scale of a human subject afflicted with multiple sclerosis comprising administering to the human subject a therapeutically effective amount of the composition described herein, or of the pharmaceutical composition described herein so as to thereby reduce the disability based on EDSS scale in the human subject.

The subject invention also provides a method of reducing lesions detected by magnetic resonance imagining (MRI) in a human subject afflicted with multiple sclerosis comprising administering to the human subject a therapeutically effective amount of the described herein, or of the pharmaceutical composition described herein so as to thereby reduce the lesions detected by MRI in the human afflicted with multiple sclerosis.

The subject invention also provides use of the composition described herein for the manufacture of a medicament for the treatment of a disease in a human subject.

The subject invention also provides use of the composition described herein and of a second agent for the manufacture of a medicament for the treatment of a disease in a human subject.

### DETAILED DESCRIPTION OF THE INVENTION

The subject invention provides a composition comprising a mixture of polypeptides in the form of a tannate salt wherein each polypeptide consists of the amino acids L-glutamic acid, L-alanine, L-tyrosine and L-Lysine, and wherein the polypeptides in the mixture do not all have the same amino acid sequence.

In an embodiment of the composition, the amino acids are present in the mixture in an amount such that the average molar fraction of amino acids is: L-glutamic acid 0.129-0.153; L-alanine 0.392-0.462; L-tyrosine 0.086-0.100; and L-lysine 0.300-0.374.

In another embodiment of the composition, the amino acids are present in the mixture in an amount such that the average molar fraction of the amino acids is: L-glutamic acid 0.141; L-alanine 0.427; L-tyrosine 0.095; and L-lysine 0.338.

In another embodiment of the composition, in the mixture the polypeptides have an average molecular weight from 2000 to 40,000 Daltons.

In another embodiment of the composition, less than 5% of the polypeptides in the mixture have a molecular weight above 40,000 Daltons.

In another embodiment of the composition, less than 2.5% of the polypeptides in the mixture have a molecular weight above 40,000 Daltons.

In another embodiment of the composition, 75% of the polypeptides have a molecular weight between 2000 and 20,000 Daltons.

In another embodiment of the composition, in the mixture the polypeptides have an average molecular weight from 4,000 to 13,000 Daltons.

In another embodiment of the composition, in the mixture the polypeptides have an average molecular weight from 4,700 to 11,000 Daltons.

In another embodiment of the composition, in the mixture the polypeptides have an average molecular weight from 5,000 to 9,000 Daltons.

In another embodiment of the composition, in the mixture the polypeptides have an average molecular weight from 4,000 to 8,600 Daltons.

In another embodiment of the composition, in the mixture the polypeptides have an average molecular weight from 4,000 to 8,000 Daltons.

In another embodiment of the composition, in the mixture the polypeptides have an average molecular weight of 6,250 to 8,400 Daltons.

In another embodiment of the composition, in the mixture the polypeptides have an average molecular weight of 7,700 Daltons.

In another embodiment of the composition, in the mixture the polypeptides have an average molecular weight of 13,500 to 18,500 Daltons.

In another embodiment of the composition, 13% to 38% of the polypeptides have a diethylamide group instead of a carboxyl group present at one end thereof.

In another embodiment of the composition, 68% of the polypeptides have a molecular weight between 7000 and 41,000 Daltons.

In another embodiment of the composition, the average molecular weight of polypeptides in the mixture is 16,000 Daltons.

In another embodiment of the composition, 19% to 28% of the polypeptides in the mixture have diethylamide at one end thereof.

In another embodiment of the composition, the remainder of polypeptides in the mixture have a carboxyl group at the C-terminus.

In another embodiment of the composition, 35-45% of the polypeptides in the mixture have a L-alanine at the N-terminus.

In another embodiment of the composition, 37-41% of the polypeptides in the mixture have an L-alanine at the N-terminus.

In another embodiment of the composition, 38-396 of the polypeptides in the mixture have an L-alanine at the N-terminus.

In another embodiment of the composition, 39% of the polypeptides in the mixture have an L-alanine at the N-terminus.

In another embodiment of the composition, less than 5% of the polypeptides in the mixture have a molecular weight below 4,700 Daltons.

In another embodiment of the composition, less than 3% of the polypeptides in the mixture have a molecular weight below 4,700 Daltons.

In another embodiment of the composition, the composition is lyophilized.

The subject invention also provides a pharmaceutical composition comprising a therapeutically effective amount of the composition described herein and a pharmaceutically acceptable carrier.

In an embodiment of the pharmaceutical composition, the polypeptide mixture is in a nanoparticle.

In another embodiment of the pharmaceutical composition, the polypeptide mixture is attached to a nanoparticle.

In another embodiment of the pharmaceutical composition, the polypeptide mixture is attached electrostatically to the nanoparticle.

In another embodiment, the pharmaceutical composition is in an enteric matrix.

In another embodiment, the pharmaceutical composition is in solid form.

In another embodiment, the pharmaceutical composition is in the form of a tablet, capsule, pill, powder or granule. In another embodiment of the pharmaceutical composition, the solid form is enterically coated.

In another embodiment, the pharmaceutical composition is in the form of a tablet.

In another embodiment of the pharmaceutical composition, the effective amount is 0.1 mg to 70 mg.

In another embodiment, the pharmaceutical composition further comprises at least one of riluzole, glatiramer acetate, baclofen, phenytoin, quinine, amitriptyline, phenothiazine, chlorpromazine, butyrophenone neuroleptics, geldanamycin, RNA interference, trehalose, cystamine, rapamycin, glucocorticoid, nonsteroidal anti-inflammatory drug, minocycline, folic acid, creatine, dichloroacetate, nicotinamide, riboflavin, carnitine, tauroursodeoxycholic acid, ginkgo biloba, coenzyme Q10, vitamin A, vitamin C, vitamin E, selenium, lipoic acid, arginine, mithramycin, remacemide, filuzole, lamotrigine, memantine, gabapentin, HDAC inhibitors, retinoic acid, reserpine, anticholinergics, diphenoxylate, loperamide, deodorized opium tincture, codeine, metronidazole, sulfasalazine, corticosteroid, azathioprine, 6-mercaptopurine, cyclosporine, T lymphocyte aphaeresis, 4-amino quinolines, methotrexate), loperamide, 5-aminosalicylic acid (5-ASA), balsalazide, olsalazine, ACTH 75, ACTH 120, antibiotic, pilocarpine, isoptocarpine timolol hemihydrate, timolol maleate, betaxolol, levobunolol, carteolol, metipranolol, epinephrine, dipivefrin, carbachol, apraclonidine, brimonidine, dorzolamide, latanoprost, travaprost, brimatoprost, brinzolamide, cholinesterase inhibitor, demecarium, isoblurophate, carbonic anhydrase inhibitor, mannitol, oral glycerin, and mydriatics, atropine, meclizine, dienhydrinate, prochlorperazine, scopolamine, diphenhydramine, clonazepam, primidone, botulinum toxin, actazolamide, and cabidopa-levodopa, isoniazid, diazepam, clonazepam, dantrolene sodium, tizanidine, clonidine, sildenafil, alprostadil, papaverine, bisacodyl, magnesium hydroxide, glycerin, psyllium hydrophilic mucilloid, sodium phosphate, anti-tumor necrosis factor (TNF), docusate, oxybutynin, desmopressin, vasopressin, tolterodine, carbamazepine, imipramine, bethane, phenoxybenzamine, terazosin, propantheline, oxybutonin, hyoscyamine, methenamine, nitrofurantoin, phenazopyridine, ciprofloxacin, amantadine, pemoline, vitamin D derivative, modafinil, fluoxetine, sertraline, venlafaxine, citalopram, parocetine, trazodone, nortriptyline, imipramine, dothiepin, lofepramine, doxepin, protriptyline, tranylcypromine, moclobemide, bupropion, nefazodone, mirtazapine, zolpidem, alprazolam, temazepam, buspirone, topiramate, zonisamide, desipramine, imipramine, doxepin, protriptyline, pentozifylline, hydroxyzine, natalizumab, steroids, muscle relaxants, prednisolone, dexamethasone, corticotrophin, immunosuppressants, acyclovir, azathioprine, cyclophosphamide, mitoxantrone, cyclosporine, methotrexate, cladribine, interferons, laquinimod, alemtuzumab, 4-aminopyridine, 3,4-diaminopyridine, eliprodil, IV immunoglobin, pregabalin, or ziconotide.

The subject invention also provides a process for making a mixture of tannate salt of polypeptides, wherein each polypeptide consists of the amino acids L-glutamic acid, L-alanine, L-tyrosine and L-lysine, and wherein the polypeptides in the mixture do not all have the same amino acid sequence, comprising:
a) obtaining a mixture of acetate salt of polypeptides, wherein each polypeptide consists of the amino acids L-glutamic acid, L-alanine, L-tyrosine and L-lysine, and wherein the polypeptides in the mixture do not all have the same amino acid sequence; and
b) contacting the mixture of acetate salt of the polypeptides of step a) with tannic acid under suitable conditions to thereby form the mixture of tannate salt of the polypeptide.

In an embodiment of the process, step b) comprises formation of a suspension and separation of the solid from the suspension.

In another embodiment, the process further comprises washing the solid from suspension with an aqueous solution of electrolyte.

In another embodiment of the process, the electrolyte is NaCl dissolved in a 10% aqueous solution.

In another embodiment of the process, the mixture of acetate salt of polypeptides in step a) is obtained by
a) polymerizing N-carboxyanhydrides of L-tyrosine, L-alanine, γ-benzyl glutamate and trifluoroacetyl lysine with a predetermined amount of diethylamine to form a mixture of protected polypeptides;
b) removing the benzyl protecting group from the protected polypeptides by contacting the polypeptides with a hydrogen bromide and acetic acid solution at a temperature in the range of 17° C. to 23° C. for a period of 7 to 18 hours to produce a mixture of trifluoroacetyl protected polypeptides;
c) removing the trifluoroacetyl protecting group from the trifluoroacetyl protected polypeptides by contacting the protected polypeptides with an organic base solution to obtain deprotected polypeptide; and
d) subjecting the deprotected polypeptides from step c) to ultrafiltration.

The subject invention also provides a method of treating a human subject afflicted with an autoimmune disease comprising administering to the subject a therapeutically effective amount of the composition described herein, or of the pharmaceutical composition described herein, so as to treat the human subject.

The subject invention also provides a method of treating a human subject afflicted with an inflammatory non-autoimmune disease, an immune mediated disease, or a disease associated with demyelination comprising administering to the human subject a therapeutically effective amount of the composition described herein, or of the pharmaceutical composition described herein, so as to treat the human subject.

The subject invention also provides a method of alleviating a symptom of an autoimmune disease in a subject afflicted with such a disease, comprising administering to the human subject the composition described herein, or of the pharmaceutical composition described herein in an amount effective to alleviate the symptom.

The subject invention also provides a method of alleviating a symptom of an inflammatory non-autoimmune disease, an immune mediated disease, or a disease associated with demyelination in a subject afflicted with such a disease, comprising administering to the human subject the composition described herein, or of the pharmaceutical composition described herein in an amount effective to alleviate the symptoms.

The subject invention also provides a method of promoting nerve regeneration or preventing or inhibiting secondary degeneration which may otherwise follow primary nervous system injury in a human subject comprising administering to the human subject a therapeutically effective amount of the composition described herein, or of the pharmaceutical composition described herein.

The subject invention also provides a method of treating a human subject afflicted with a neurodegenerative disease comprising administering to the human subject a therapeutically effective amount of the composition described herein, or of the pharmaceutical composition described herein so as to thereby treat the human subject.

The subject invention also provides a method of alleviating a symptom of an neurodegenerative disease comprising administering to the human subject the composition described herein, or of the pharmaceutical composition described herein in an amount effective to alleviate the symptom.

In an embodiment of the method, the neurodegenerative disease is Huntington's disease.

In another embodiment, the method further comprises administering to the subject a second agent, wherein the second agent is phenothiazine, butyrophenone neuroleptics, haloperidol, reserpine, or a combination thereof.

In another embodiment of the method, the neurodegenerative disease is glaucoma.

In another embodiment of the method, the method preserves the structural integrity of the optic nerve of the human subject afflicted with glaucoma.

In another embodiment of the method, the method preserves the retinal cells in the human subject afflicted with glaucoma.

In another embodiment of the method, the method reduces the rate of visual field loss in the human subject afflicted with glaucoma.

In another embodiment, the method further comprises administering to the subject of a second agent, wherein the second agent is glatiramer acetate, pilocarpine, timolol maleate, betaxolol, levobunolol, metipranolol, epinephrine, dipivefrin, carbachol, potent cholinesterase inhibitors, carbonic anhydrase inhibitors, atropine, mydriatics, or a combination thereof.

In another embodiment, the method further comprises laser trabeculoplasty, filtering surgery, peripheral iridectomy, or laser iridectomy.

In another embodiment of the method, the administration is through an intravenous, intraperitoneal, intramuscular, subcutaneous, oral, intranasal, buccal, vaginal, rectal, intraocular, intrathecal, topical or intradermal route.

The subject invention also provides a method of treating a human subject afflicted with an inflammatory bowel disease comprising administering to the human subject a therapeutically effective amount of the composition of any one ofdescribed herein, or of the pharmaceutical composition described herein so as to treat of the inflammatory bowel disease.

The subject invention also provides a method of alleviating a symptom of an inflammatory bowel disease comprising administering to the human subject the composition described herein, or of the pharmaceutical composition described herein in an amount effective to alleviate the symptom.

In another embodiment, the method further comprises administering to the subject a second agent, wherein the second agent is an anticholinergic, diphenoxylate, loperamide, deodorized opium tincture, codeine, antibiotics, metronidazole, sulfasalazine, corticosteroids, prednisone, hydrocortisone, antimetabolites, azathioprine, 6-mercaptopurine, cyclosporine, methotrexate, 4-amino quinolines, loperamide, 5-aminosalicylic acid (5-ASA), sulfasalazine, olsalazine, prednisone, ACTH 75, ACTH 120, antibiotics, or a combination thereof.

In another embodiment, the method further comprises ingestion of an elemental diet, hyperalimentation, surgery, proctoclectomy with abdominoperineal resection, emergency colectomy, subtotal colectomy with ileostomy or rectosigmoid mucous fistula.

In another embodiment of the method, the inflammatory bowel disease is Crohn's Disease.

In another embodiment of the method, the inflammatory bowel disease is ulcerative colitis.

In another embodiment of the method, the administration of the composition is through an intravenous, intraperitoneal, intramuscular, subcutaneous, oral, intranasal, buccal, vaginal, rectal, intraocular, intrathecal, topical or intradermal route.

The subject invention also provides a method of treating a human subject afflicted with multiple sclerosis comprising administering to the human subject a therapeutically effective amount of the composition described herein, or of the pharmaceutical composition described herein so as to thereby treat the human subject afflicted with multiple sclerosis.

The subject invention also provides a method of alleviating a symptom of multiple sclerosis in a human subject afflicted with multiple sclerosis comprising administering to the human subject the composition described herein, or of the pharmaceutical composition described herein in an amount effective to alleviate the symptom of multiple sclerosis.

The subject invention also provides a method of reducing the frequency of relapses in a human subject afflicted with relapse remitting multiple sclerosis comprising administering to the human subject a therapeutically effective amount of the composition described herein, or of the pharmaceutical composition described herein so as to thereby reduce the frequency of relapses in the human subject.

The subject invention also provides a method of reducing the disability based on the EDSS scale of a human subject afflicted with multiple sclerosis comprising administering to the human subject a therapeutically effective amount of the composition described herein, or of the pharmaceutical composition described herein so as to thereby reduce the disability based on EDSS scale in the human subject.

The subject invention also provides a method of reducing lesions detected by magnetic resonance imagining (MRI) in a human subject afflicted with multiple sclerosis comprising administering to the human subject a therapeutically effective amount of the composition described herein, or of the pharmaceutical composition described herein so as to thereby reduce the lesions detected by MRI in the human afflicted with multiple sclerosis.

In an embodiment, the method further comprises administration of a second agent, wherein the second agent is glatiramer acetate, a pain reliever, a steroid, a muscle relaxant, prednisone, dexamethasone, an immunosuppressant, azathioprine, cyclophosphamide, an interferon, natalizumab, riluzole, alphacalcidol, calcitriol, rasagiline, minocycline, mitoxantrone, simvastatin, or a combination thereof.

In another embodiment of the method, the amount of the composition and the dose of the second agent taken together are effective to treat the subject.

In another embodiment of the method, each of the amount of the composition taken alone, and the dose of the second agent taken alone is effective to treat the subject.

In another embodiment of the method, either the effective amount of the composition taken alone, the dose of the second agent taken alone is not effective to treat the subject.

In another embodiment of the method, the subject never previously received the second agent for treatment of the condition.

In another embodiment of the method, the subject has received the second agent for therapy, but is no longer receiving the second agent for treatment of the condition.

In another embodiment of the method, the amount of the mixture of polypeptides in the tannate salt form is 0.1 mg to 100 mg.

In another embodiment of the method, the amount of the mixture of polypeptides in the tannate salt form is 0.1 mg to 1000 mg/day.

In another embodiment of the method, the administration is through an intravenous, intraperitoneal, intramuscular, subcutaneous, oral, intranasal, buccal, vaginal, rectal, intraocular, intrathecal, topical or intradermal route.

In another embodiment of the method, the composition is administered orally.

The subject invention also provides the composition described herein, or the pharmaceutical composition described herein, for use as a medicament.

The subject invention also provides a product containing the composition described herein and a second pharmaceutical agent, as a combined preparation for simultaneous, separate or sequential use as a medicament.

The subject invention also provides use of the composition described herein for the manufacture of a medicament for the treatment of a disease in a human subject.

The subject invention also provides use of the composition described herein and of a second agent for the manufacture of a medicament for the treatment of a disease in a human subject.

In any one of the disclosed embodiments, the polypeptides in the mixture do not all have the same amino acid sequence.

In any one of the disclosed embodiments, the effective amount may be 0.1 mg to 70 mg. In an embodiment the effective amount may be 0.5 mg to 60 mg; 1 mg to 50 mg; 5 mg to 35 mg; 10 mg to 30 mg; 45 mg to 70 mg; 50 mg to 70 mg; 15 mg to 25 mg; 18 mg to 22 mg; 0.1 mg to 2 mg; 0.5 mg to 1.5 mg; 2 mg to 7 mg; 4 mg to 6 mg; 12 mg to 18 mg; 14 mg to 16 mg; 17 mg to 23 mg; 19 mg to 21 mg; 27 mg to 33 mg; 29 mg to 31 mg; 47 mg to 53 mg; or 49 mg to 51 mg.

In any one of the disclosed embodiments, the composition may have a pH between 5.5 and 9.0; between 5.5 and 8.5; between 5.5 and 7.5; between 5.5 and 7.0; between 5.5 and 6; may be 5.7; or may be 5.5.

In any one of the disclosed embodiments, said pharmaceutical composition is in solid form, liquid form, aerosol or inhalable powder.

In any one of the disclosed process embodiments, the hydrogenolysis catalyst is Palladium/carbon, Raney Nickel, Pt, Pt/C. PtO₂, Pd(OH)₂, Rh/C, or RhCl(PPh₃)₃. In a further embodiment, the hydrogenolysis catalyst is Palladium/carbon. In a further embodiment, the weight ratio of protected polypeptides to palladium/carbon catalyst is 10:1.

In any one of the disclosed process embodiments, the step of contacting the polypeptides with the hydrogenolysis catalyst is performed in a solvent selected from the group consisting of methanol, ethanol or isopropanol. In a further embodiment, the solvent is methanol.

In any one of the disclosed process embodiments, the initiator is a primary amine, a dialkyl amine or sodium methoxide. In a further embodiment, the initiator is diethylamine.

In any one of the disclosed process embodiments, the amount of initiator is 1% to 10% by weight. In a further embodiment, the amount of initiator is 2% to 5% by weight. In another embodiment, the amount of initiator is 2% by weight. In a further embodiment, the amount of initiator is 5% by weight.

In any one of the disclosed process embodiments, the organic base is an aqueous organic base. In a further embodiment, the aqueous organic base is a primary, secondary or tertiary amine or methanolic ammonia. In another embodiment, the aqueous organic base is piperidine.

In any one of the disclosed process embodiments, the amount of initiator may be 0.05% to 19% by weight or 0.1% to 17% by weight or 0.5% to 15% by weight or 1% to 10% by weight or 2% to 5% by weight or 2% by weight or 5% by weight.

In any one of the disclosed process embodiments, the first peak molecular weight or the desired peak molecular weight may be 2000 Daltons to 40,000 Daltons or 2000 Daltons to 20,000 Daltons or 4000 Daltons to 8600 Daltons or 4000 Daltons to 8000 Daltons or 6250 Daltons to 8400 Daltons or 2000 Daltons to 13,000 Daltons or 4700 Daltons to 13,000 Daltons or 10,000 Daltons to 25,000 Daltons or 15,000 Daltons to 25,000 Daltons or 18,000 Daltons to 25,000 Daltons or 20,000 Daltons to 25,000 Daltons or 13,000 Daltons to 18,000 Daltons or 15,000 Daltons or 12,500 Daltons.

In any one of the disclosed process embodiments, the organic base may be an aqueous organic base. In another embodiment, the aqueous organic base may be a primary, secondary or tertiary amine or methanolic ammonia. In yet another embodiment, the aqueous organic base may be piperidine.

In any one of the disclosed process embodiments, the removal of the benzyl protecting group in step b) is performed at a temperature in the range of 17-21° C. In a further embodiment, the removal of the benzyl protecting group in step b) is conducted at a temperature in the range of 19-20° C. In a further embodiment, the removal of the benzyl protecting group in step b) is conducted over a period of 7 to 15 hours. In a further embodiment, the removal of the benzyl protecting group in step b) is conducted over a period of approximately 15 hours. In a further embodiment, the organic base in step c) is a primary amine, a secondary amine, a tertiary amine, or methanolic ammonia. In a further embodiment, the organic base is piperidine. In a further embodiment, the hydrogen bromide and acetic acid solution is from 10% to 36% hydrobromic acid in acetic acid. In a further embodiment, the hydrogen bromide and acetic acid solution is 33% hydrobromic acid in acetic acid. In a further embodiment, the hydrogen bromide and acetic acid solution is pretreated with a bromine scavenger in order to remove free bromine. In a further embodiment, the bromine scavenger is phenol. In a further embodiment, the process further comprises a step of lyophilizing the composition.

To obtain a proper mixture of polypeptides in the form of an acetate salt, the process may further comprise obtaining a batch of a mixture of acetate salt of polypeptides; determining the average molecular weight of the mixture of polypeptides in the batch using a molecular weight-calibrated gel permeation chromatography column; and
including in the pharmaceutical product the mixture if the mixture is determined to have an average molecular weight between 2000 and 40,000 Daltons,
wherein the gel permeation chromatography column is calibrated by subjecting a plurality of molecular weight markers to chromatography on the column to establish a relationship between the retention time on the column and molecular weight, wherein each of the markers is a polypeptide consisting essentially of alanine, glutamic acid, tyrosine and lysine and has a predetermined amino sequence. In a further embodiment, in the mixture the molar fraction of alanine is 0.427, of glutamic acid is 0.141, of lysine is 0.337 and of tyrosine is 0.093. In a further embodiment, the gel permeation chromatography column comprises a cross-linked agarose-based medium, with an exclusion limit of 2×10⁶ Daltons, an optimal separation range of 1000 to 3×10⁵ Daltons, and a bead diameter of 20-40 μm. In a further embodiment, in the molecular weight markers the molar fraction of alanine is 0.38 to 0.5, of glutamic acid is 0.13 to 0.15, of tyrosine is 0.08 to 0.10 and of lysine is 0.3 to 0.4. In a further embodiment, in the molecular weight markers the molar fraction of alanine is 0.422 to 0.444, of glutamic acid is 0.133 to 0.143, of tyrosine is 0.086 to 0.093 and of lysine is 0.333 to 0.349. In a further embodiment, one or a plurality of the molecular weight markers is selected from the group consisting of

(SEQ ID NO: 1)

AKKYAKKEKAAKKAYKKEAKAKAAEAAAKEAAYEA;

(SEQ ID NO: 2)

AKKYAKKAKAEKAKKAYKAAEAKKAAKYEKAAAEKAAAKEAAYEA;

(SEQ ID NO: 3)

AKKYAKKEKAYAKKAEKAAKKAEAKAYKAAEAKKKAEAKYKAEAAKAAAK
EAAYEA;

(SEQ ID NO: 4)

AKKYAKKEKAYAKAKKAEAKAAKKAKAEAKKYAKAAKAEKKEYAAAEAKY
KAEAAKAAAKEAAYEA;

(SEQ ID NO: 5)

AKKYAKKEKAYAKKAEKAAKKAEAKAYKAAEAKKKAKAEAKKYAKAAKAE
KKEYAAAEAKYKAEAAKAAAKEAAYEA;

(SEQ ID NO: 6)

AKKYAKKEKAYAKKAEKAAKKAEAKAYKAAEAKKKAKAEAKKYAKAAKAE
KKEYAAAEAKYKAEAAKKAYKAEAAKAAAKEAAYEA;
and

(SEQ ID NO: 7)

AKKYAKKAEKAYAKKAKAAKEKKAYAKKEAKAYKAAEAKKKAKAEAKKYA
KEAAKAKKEAYKAEAKKYAKAAKAEKKEYAAAEAKKAEAAKAYKAEAAKA
AAKEAAYEA,

wherein A represents alanine, K represents lysine, Y represents tyrosine, and E represents glutamic acid.

In yet a further embodiment, the plurality of molecular weight markers may comprise five polypeptides having the following sequences:

(SEQ ID NO: 8)

AEKYKAKKAKEKAYKKKAKEAKKAKYKAKEAKAYKAEKKAKYAKAKEKAY
AKAKEAKAYAKAKAKAEKAKAKAKYAEKAKAAKYAEKAAKYAEAKAKAAE
AKYAAEAKEAAKAAEAKYAAKAEAAKYAAEKAAEKYAKAEAAAEAKEAA;

(SEQ ID NO: 9)

AKKKYKAKEKKAKKKAKEKKYKAKKAKYKEKAAKYKAKKAKAKYKAKAEK
AKAKAEKAKAYAEKAKAKYAKEAKKYAEKAKKAEYKAKEAAEKAKAYAKE
AAKAEKAAKAAEKAAKAYAKAEAAAKEAAYAAKAEAKAAYAAEAAKAEYA
AEAAKEAAYAAAEYAAEAA;

(SEQ ID NO: 10)

AKKKYKAKEKKAKKKAKEKKKYKAKEKKAKKYKEKAAKYKAKKAKKEAAK
YKKAKAEKAKYAKEKAEKAKAYAEKAKAKYAKEAKKYAEKAKKAEYKAKE
AAEKAKAYAKEAAKAEAKAAKYAAEKAAEAAKAAYAKAEAAKAAKEAAYA
AKAEAAKAAYAAEAAKAEYAAEAAKEAAYAAAEYAAAEAA;

(SEQ ID NO: 11)

AKKKYKAKEKKAKAKKKAKEKKKYKAKKEKKAKKYKEKAAKYKAKKEKAK
KEAAKYKKAKKYKAEKAKYAKEKAKEKAKAYAEKAEKAAKYAAKEAKKYA
EKAAEKKAEYKAKEAAEKYAAKAYAAKEAAKAYKEAKAAKYAKAAEKAAA
KEAAKAAYAAKAEAAKEAAKEAAYAAAKAEAAAAKAAAYAAEAAAKAEYA
AEAAAKEAAYAAAEYAAAEAA;
or

(SEQ ID NO: 12)

AKKKYKAKEKKAKYKAKKEKKAKEKKAKKKYKAKKKAAEKKYAKEKKAKE
KAAKKYKAKKEKAKKEAAKYKKAKKYKAEKKAKYAAKEKAKEKAKAYAEK
KAEKAAKYAAKEAKKYAEKAAEKKAEYKAKEAAEKYAAKKAEYAAEKEAA
KAYKEAKAAKYAKAAEKAKAAKEAAKAAYAAKAEAAKEAAKEAAYAAAKA
EAAAYAAAKAAAAYAAEAAAKAEYAAAEAAAKEAAYAAAEYAAAAEAAA,

wherein A represents L-alanine, K represents L-lysine, Y represents L-tyrosine, and E represents L-glutamic acid.

In a further embodiment, the process further comprises a step of lyophilizing of the mixture having the average molecular weight between 2000 to 40,000 Daltons.

In any one of the disclosed process embodiments, the solution of hydrobromic acid in acetic acid comprises less than 0.1% of free bromine. In another embodiment, the solution of hydrobromic acid in acetic acid comprises less than 0.05% of free bromine. In a further embodiment, the solution of hydrobromic acid in acetic acid comprises less than 0.01% of free bromine. In yet another embodiment, the solution of hydrobromic acid in acetic acid comprises less than 0.001% of free bromine. In a further embodiment, the solution of hydrobromic acid in acetic acid is free of free bromine.

In any one of the disclosed process embodiments, the solution of hydrobromic acid in acetic acid comprises less than 1000 ppm of metal ion impurities. In yet another embodiment, the solution of hydrobromic acid in acetic acid comprises less than 500 ppm of metal ion impurities.

In one embodiment, the solution of hydrobromic acid in acetic acid comprises less than 100 ppm of metal ion impurities. In another embodiment, the solution of hydrobromic acid in acetic acid comprises less than 30 ppm of metal ion impurities. In yet another embodiment, the solution of hydrobromic acid in acetic acid comprises less than 20 ppm of metal ion impurities. In a further embodiment, the solution of hydrobromic acid in acetic acid comprises less than 10 ppm of metal ion impurities. In another embodiment, the solution of hydrobromic acid in acetic acid is free of metal ion impurities.

In any one of the disclosed process embodiments, the hydrogen bromide and acetic acid solution is from 10% to 36% hydrobromic acid in acetic acid. In another embodiment, the hydrobromic acid in acetic acid is from 16% to 33% hydrobromic acid in acetic acid; 18% to 33% hydrobromic acid in acetic acid; 20% to 37% hydrobromic acid in acetic acid; 20% to 33% hydrobromic acid in acetic acid; 22% to 33% hydrobromic acid in acetic acid; 24% to 33% hydrobromic acid in acetic acid; 25% to 35% hydrobromic acid in acetic acid; 26% to 33% hydrobromic acid in acetic acid; 28% to 33% hydrobromic acid in acetic acid; 30% to 34% hydrobromic acid is acetic acid; 30% to 33% hydrobromic acid in acetic acid; or 32% to 33% hydrobromic acid in acetic acid. In a further embodiment, the solution is 33% hydrobromic acid in acetic acid. In another embodiment, the solution is 16% hydrobromic acid in acetic acid. The hydrogen bromide and acetic acid solution may be pretreated with a bromine scavenger, such as phenol, in order to remove free bromine. In a further embodiment, the solution is produced in a non-metallic reactor. In another embodiment, the solution is prepared in a glass-lined or Teflon lined reactor. In yet another embodiment, the color of the hydrobromic acid in acetic acid solution is less than 2000 APHA. In a further embodiment, the color of the hydrobromic acid in acetic acid solution is less than 1000 APHA. In another embodiment, the color of the hydrobromic acid in acetic acid solution is less than 700 APHA. In yet another embodiment, the color of the hydrobromic acid in acetic acid solution is less than 500 APHA.

When the neurodegenerative disease to be treated is glaucoma, the composition may be administered once every 1 to 12 weeks; once every 3 to 12 weeks; once every 3 to 8 weeks; once every 2 to 6 weeks; once every 1 to 2 weeks; once every 3 to 5 weeks; once every 4 to 10 weeks; once every 4 weeks; once every 2 months.

In any one of the disclosed embodiments, the composition may be administered intranasally and the dose may be less than 1 mg or the composition may be administered orally and the dose may be 70 mg. Preferably, the composition is administered by injection.

In any one of the disclosed embodiments, the composition may be administered every 1 to 60 days; every 1 to 30 days; every 5 to 60 days; every 7 to 60 days; every 5 to 30 days; every 20 to 40 days; every 50 to 60 days; every 5 to 9 days; every 6 to 8 days; every 7 days; every 14 days; 30 days; 60 days; or every 2 months.

When the neurodegenerative disease is multiple sclerosis, the method may further comprise plasmaphoresis, or total lymphoid radiation.

In any one of the disclosed embodiments, the amount of the composition may be 0.1 mg to 100 mg of the composition; 1 mg to 80 mg of the composition; 1 mg to 50 mg of the composition; 5 mg to 25 mg of the composition; 25 mg to 75 mg of the composition; 2 mg to 8 mg of the composition; 4 mg to 6 mg of the composition; 12 mg to 18 mg of the composition; 14 mg to 16 mg of the composition; 27 mg to 33 mg of the composition; 29 mg to 31 mg of the composition; 47 mg to 53 mg of the composition; 49 mg to 51 mg of the composition; 5 mg of the composition; 15 mg of the composition; 30 mg of the composition; or 50 mg of the composition.

Therapeutic Uses

Based on the data gathered, the mixture of polypeptides in the form of a tannate salt of the invention is contemplated for use in treating at least the same conditions as glatiramer acetate has been disclosed to treat. Specific diseases and classes of diseases are discussed below.

An autoimmune disease or disorder is one where the immune system produces autoantibodies to an endogenous antigen with consequent injury to tissues (Merck Manual of Diagnosis and Therapy (1999), Merck Research Laboratories, (Whitehouse Station, N.J.), 1061). These diseases may be either cell-mediated disease (e.g. T-cell) or antibody-mediated (e.g. B cell) disorders (U.S. Patent Application Publication No. 2002/0055466 A1, published May 9, 2002 (Aharoni, et al.)). Autoimmune diseases are contemplated for treatment with the composition comprising the mixture of polypeptides of the invention.

Specific autoimmune diseases contemplated for treatment with the composition comprising the mixture of polypeptides of the invention are polyarthritis, juvenile arthritis, Felty's syndrome, autoimmune hemolytic anemia, autoimmune oophoritis, autoimmune thyroiditis, autoimmune uveoretinitis, Crohn's disease, ulcerative colitis such as in inflammatory bowel disease, chronic immune thrombocytopenic purpura, contact sensitivity disease, diabetes mellitus, Graves disease, Guillain-Barre's syndrome, Hashimoto's disease (thyroiditis), idiopathic myxedema, myasthenia gravis, psoriasis, pemphigus vulgaris, rheumatoid arthritis, uveitis, lupus nephritis, CNS lupus or systemic lupus erythematosus. GA has been disclosed for use in the treatment of these diseases in, e.g. U.S. Patent Application Publication No. 2002/0055466 A1, published May 9, 2002 (Aharoni, et al.); U.S. Pat. No. 6,514,938 B1, issued Feb. 4, 2003 to Gad, et al.; PCT International Publication No. WO 01/60392, published Aug. 23, 2001 (Gilbert, et al.); U.S. Patent Application Publication No. 2004/0006022, published Jan. 8, 2004 (Strominger, et al.).

Inflammatory, non-autoimmune diseases are diseases which impact the central nervous system, but do not include an autoimmune component and are associated with an inflammatory response in the subject afflicted with the disease. Inflammatory, non-autoimmune diseases are contemplated for treatment with the composition comprising the mixture of polypeptides of the invention. Specific inflammatory, non-autoimmune diseases contemplated for treatment with the polypeptide mixtures of the invention are Alzheimer's disease, Parkinson's disease, HIV encephalopathy, brain tumor, glaucoma, neuropathy, dementia, central nervous system infection, central nervous system bacterial infection, meningitis, stroke, and head trauma. GA has been disclosed for use in the treatment of these diseases in, e.g. U.S. Patent Application Publication No. 2002/0077278 A1, published Jun. 20, 2002 (Young, et al.).

The composition of the invention is also contemplated to be useful to promote nerve regeneration or to prevent or inhibit secondary degeneration which may otherwise follow primary nervous system (NS) injury, e.g., closed head injuries and blunt trauma, such as those caused by participation in dangerous sports, penetrating trauma, such as gunshot wounds, hemorrhagic stroke, ischemic stroke, glaucoma, cerebral ischemia, or damages caused by surgery such as tumor excision.

In addition, the composition of the mixture may be used to ameliorate the effects of disease that result in a degenerative process, e.g., degeneration occurring in either gray or white matter (or both) as a result of various diseases or disorders (such as neurodegenerative diseases), including, without limitation: diabetic neuropathy, senile dementias, Alzheimer's disease, Parkinson's Disease, Huntington's disease, uveitis, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, obstructive lung disease, chronic ataxic neuropathy, ophthalmic neuropathy, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease, lipoproteinemia, epilepsy, hyperalgesia, psychosis, seizures, abnormally elevated intraocular pressure, oxidative stress, opiate tolerance and dependence. Multiple sclerosis is not considered a neurodegenerative disease in this disclosure, but rather a demyelinating disease. In addition, mixtures of this invention are contemplated to be useful for their glutamate protective aspect, i.e. for injury or disease caused or exacerbated by glutamate toxicity, for example, post-operative treatments generally, and surgical tumor removal from the central nervous system (CNS). GA has been disclosed for use in the treatment of these diseases in, e.g. U.S. Patent Application Publication No. 2002/0037848 A1, published Mar. 28, 2002 (Eisenbach-Schwartz) and U.S. Patent Application Publication No. 2003/0004099 A1, published Jan. 2, 2003 (Eisenbach-Schwartz).

Certain immune-mediated diseases contemplated for treatment with a composition comprising the polypeptide mixture of the invention are characterized by undesirable immune hypersensitivity to one or more antigens and include host-versus-graft disease (HVGD) and graft-versus-host disease (GVHD), which are exemplified, respectively, by graft rejection by the host immune system and by attack on the host by donor T cells. These diseases are a significant barrier to transplantation systems such as organ transplantations and bone marrow reconstitutions. Other immune mediated diseases that are contemplated for treatment by the polypeptide mixture of the invention include delayed-type hypersensitivity (DTH) which is associated with contact antigens such as poison ivy and poison oak and various chemicals, as well as tuberculosis, leprosy, leishmaniasis, deep fungal infections, etc. GA has been disclosed for use in the treatment of these diseases in, e.g. U.S. Pat. No. 6,514,938 B1, issued Feb. 4, 2003 to Gad, et al.; and PCT International Publication No. WO 01/60392, published Aug. 23, 2001 (Gilbert, et al.); PCT International Publication No. WO 00/27417, published May 19, 2000 (Aharoni, et al.).

Polypeptide mixtures of the invention are also contemplated as a treatment for diseases associated with demyelination of central nervous system axons such as multiple sclerosis, acute disseminated encephalomyelitis, transverse myelitis, demyelinating genetic diseases, spinal cord injury, virus-induced demyelination, Progressive Multifocal Leucoencephalopathy, Human Lymphotrophic T-cell Virus I (HTLVI)-associated myelopathy, and nutritional metabolic disorders such as vitamin B12 deficiency and central pontinemyelinolysis. GA has been disclosed for use in the treatment of these diseases in, e.g. PCT International Publication No. WO 01/97846, published Dec. 27, 2001 (Moses, et al.)

Methods of Administration

Methods of administration include all standard methods, e.g. by parenteral, intravenous, intraperitoneal, intramuscular, subcutaneous, mucosal, oral, intranasal, buccal, vaginal, rectal, intraocular, intrathecal, topical, transdermal and intradermal routes. Administration can be systemic or local.

For oral administration, the pharmaceutical preparation of the polypeptide mixture may be in liquid form, for example, emulsions, microemulsions, solutions, suspensions, syrups and elixirs, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). Inert diluents commonly used in the art, include, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the polypeptide mixture. For buccal administration, the compositions may take the form of tablets or lozenges formulated in conventional manner. The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

In one embodiment, the oral composition is enterically-coated. Use of enteric coatings is well known in the art. For example, Lehman teaches enteric coatings such as Eudragit S and Eudragit L. (Lehman, K., “Acrylic Coatings in Controlled Realse Tablet Manufacturer”, Manufacturing Chemist and Aerosol News, p. 39 (1973)) The Handbook of Pharmaceutical Excipients, 2.sup.nd Ed., also teaches Eudragit S and Eudragit L applications. One Eudragit which may be used in the present invention is L30D55.

Pharmaceutical compositions comprising the mixture of polypeptides of the invention may optionally be administered with an adjuvant in the usual manner for immunization. Non-limiting examples of such adjuvants include alum and incomplete Freund's adjuvant. Other manners of improving the immunogenicity of the administered peptide or polypeptide include administration in the form of an aggregation or a complex with albumin or with other carriers, all as are well known to those of ordinary skill in the vaccine art. Metabolizable lipid emulsions, such as Intralipid or Lipofundin may also be used as vehicles for the therapy in the manner disclosed in PCT International Publication No. WO 97/02016, published Jan. 23, 1997 (Cohen et al), the entire contents of which being hereby incorporated herein by reference.

The mixture of polypeptides can also be administered in the form of liposome delivery systems, such as small unilamellar vesicles, large unilamallar vesicles, and multilamellar vesicles. Liposomes can be formed from a variety of phospholipids, such as phosphatidylcholines, or lipids, such as cholesterol and stearylamine. The compounds may be administered as components of tissue-targeted emulsions.

Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmity-spermine and dioleoyl phosphatidylethanol-amine (DOPE)(GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA and the neutral lipid DOPE (GIBCO BRL).

The mixture of polypeptides disclosed in this application, having a net positive charge, can be attached electrostatically to charged nanoparticles or nanoparticle by mixing an aqueous solution of the polypeptide mixture of the invention with a suspension of the nanoparticles or microparticles. The suspension thus formed of the polypeptide mixture attached to the nanoparticles or nanoparticle can be lyophilized to a powder for long-term storage. The lyophilized powder can be reconstituted in buffer to re-obtain the suspension of drug. Suspensions of attached drug thus obtained are particularly suited for oral delivery. If made with particles having an average diameter below 200 nm the suspension is suitable for sublingual delivery since nanoparticles can transverse the sublingual membrane. For oral delivery to the gastrointestinal tract larger nanoparticles can be used since they are the size most readily recognized by the Peyer's patches and M-cells. For such oral delivery a nano-suspension, as a lyophilized powder or as a reconstituted suspension, may be delivered to the small intestine by using an enteric coated capsule. The enhanced stability of the peptide or protein when attached in a nano-suspension formulation allows for more time for the peptide drug to be absorbed in the intestine before it is degraded by enzymes in the gastrointestinal tract. Production of nanoparticles can be achieved by methods well known in the art. An example of a nanoparticle involving glatiramer acetate is described in PCT International Publication No. WO 2005/041933.

For intraocular administration the polypeptide mixture may be formulated into pharmaceutical compositions with pharmaceutically acceptable carriers, such as water or saline and may be formulated into eye drops.

The polypeptide mixture may also be formulated in rectal compositions such as suppositories or retention enemas, e.g., containing conventional suppository bases such as cocoa butter or other glycerides.

For administration by inhalation, the polypeptide mixture according to the present invention is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

The mixture of polypeptides of the invention may also be administered nasally in certain of the above-mentioned forms by inhalation or nose drops. Furthermore, oral inhalation may be employed to deliver the mixture of polypeptides of the invention to the mucosal linings of the trachea and bronchial passages.

The invention also provides a pharmaceutical pack or kit comprising one or more containers filled with one or more of the ingredients of the pharmaceutical compositions of the invention.

Combination Therapies with the Polypeptide Mixture

In various embodiments, the claimed methods can encompass the administration of a therapeutically effective amount of the polypeptide mixture of the invention alone, or in combination with another therapeutic or prophylactic agent. By administration in combination, it is meant that the polypeptide mixture of the invention can be administered either substantially simultaneously with the second agent, or that the second agent can be administered in a stepwise fashion with the polypeptide mixture of the invention. Thus, in various embodiments, depending on the particular treatment regime chosen by the physician, one may administer the polypeptide mixture of the invention at the same time as the second agent, or in other embodiments, the polypeptide mixture of the invention and the second agent can be administered hours, days, or possibly even weeks apart. Alternatively, the polypeptide mixture of the invention and the second agent are administered together for a period of time, after which, administration of the second agent is discontinued while administration of the polypeptide mixture of the invention is continued. The desired treatment regime can be determined by one skilled in the art depending upon the particulars of the patient being treated, and the desired outcome.

Furthermore, in various embodiments depending on the particular treatment regime chosen by the physician, one may administer the polypeptide mixture of the invention and the second agent, when administered in a combination as described above, in lower dosages as determined by one skilled in the art. Any therapeutic or prophylactic agent useful in the treatment of the diseases for which the polypeptide mixture of the invention may be used can be the second agent according to this invention.

The polypeptide mixture of the invention can be used together with the second agent from the inception of the treatment of the patient or the mixture can be added to a treatment regimen after the patient has already been receiving the second agent for some time. Likewise, the second agent can be added to the treatment regimen after the patient received the mixture for some time. Alternatively, the mixture can be used to replace an agent when, for example, the patient's response to the agent deteriorates or when the patient experiences side effects using the other agent. For the treatment of multiple sclerosis or its symptoms, the second agent may be glatiramer acetate (COPAXONE®), natalizumab (TYSABRI®), steroids, muscle relaxants, oral prednisone (DELTASONE®), methylprednisolone (DEPO-MEDROL®, SOLU-MEDROL®), prednisolone (DELTA-CORTEF®), dexamethasone (DECADRON®, TOBRADEX®, AK-TROL®, DEXPAK, MEDROL®), adreno-corticotrophic hormone (ACTH) (ACTHAR®), corticotrophin, immunosuppressants, acyclovir, azathioprine (IMURAN®), cyclophosphamide (CYTOXAN®, NEOSAR®), mitoxantrone (NOVANTRONE®), cyclosporine (SANDIMMUNE®), methotrexate, cladribine (LEUSTATINE®), interferons (AVONEX®, BETASERON®, BETAFERON®, REBIF®), laquinimod, ginkgo biloba, natalizumab (ANTEGREN®), alemtuzumab (CAMPATH®-1H), 4-aminopyridine (FAMPRIDINE), 3,4-diaminopyridine, eliprodil, IV immunoglobin (GAMMAGARD®, GAMMAR®-IV, GAMIMUNE® N, IVEEGAM®, PANGLOBULIN®, SANDOGLOBULIN®, VENOGLOBULIN®), ANERGIX®-MS, pregabalin, or ziconotide.

For the treatment of pain and/or altered sensation (dysaesthesia) related to multiple sclerosis, the second agent may be carbamazepine (TEGRETOL®, EPITOL®, ATRETOL, CARBATROL®), gabapentin (NEURONTIN®), topiramate (TOPAMAX®), zonisamide (ZONEGRAN®), phenytoin (DILANTIN®), desipramine (NORPRAMIN®), amitriptyline (ELAVIL®), imipramine (TOFRANIL®, IMAVATE, JANIMINE), doxepin (SINEQUAN®, ADAPIN, TRIADAPIN, ZONALON®), protriptyline (VIVACTIL®), pentozifylline (TRENTAL®), ibuprophen (ADVIL®, MOTRIN®), asprin, acetaminophen, or hydroxyzine (ATARAX®).

For the treatment of depression, anxiety, and/or insomnia related to multiple sclerosis, the second agent may be fluoxetine (PROZAC®), sertraline (ZOLOFT®, LUSTRAL®), venlafaxine (EFFEXOR XR®), citalopram (CELEXA®), parocetine (PAXIL®, SEROXAT), trazodone (DESYREL®, TRIALODINE), amitriptyline (ELAVIL®), nortriptyline (PAMELOR®, AVENTYL®), imipramine (TOFRANIL®, IMAVATE, JANIMINE), dothiepin (PROTHIADEN), lofepramine (GAMANIL), doxepin (SINEQUAN®, ADAPIN, DRIADAPIN, ZONALON®), protriptyline (VIVACTIL®), tranylcypromine (PARNATE®), moclobemide (MANERIX, AURORIX), bupropion (WELLBUTRIN SR®, AMFEBUTAMONE), nefazodone (SERZONE®), mirtazapine (REMERON®), zolpidem (AMBIEN®), alprazolam (XANAX®), temazepam (RESTORIL®), diazepam (VALIUM®), or buspirone (BUSPAR®).

For the treatment of fatigue related to multiple sclerosis, the second agent may be amantadine (SYMMETREL®), pemoline (CYLERT®), vitamin D derivatives such as alphacalcidol and calcitrol, or modafinil (PROVIGIL®).

For the treatment of urinary problems related to multiple sclerosis, the second agent may be oxybutynin (DIPTROPAN XL®), desmopressin (DDAVP®), vasopressin, tolterodine (DETROL®), carbamazepine (TEGRETOL®, EPITOL®, ATRETOL, CARBATROL®), imipramine (TOFRANIL®), bethane (URECHOLINE®), phenoxybenzamine (DIBENZYLINE®), terazosin (HYTRIN®), propantheline (PRO-BANTHINE), oxybutonin (DITROPAN®), hyoscyamine (URISPAS®, CYSTOPAS), baclofen (LIORESAL®), diazepam (VALIUM®), methenamine (HIPREX®, MANDELAMINE®), nitrofurantoin (MACRODANTIN®), phenazopyridine (PYRIDIUM®), or ciprofloxacin (CIPRO®).

For the treatment of psuedobulbar affect related to multiple sclerosis, dextromethorphan (NEURODEX).

For the treatment of bowel problems related to multiple sclerosis, the second agent may be bisacodyl (DULCOLAX®, BISACOLAX), magnesium hydroxide (milk of magnesia), glycerin (SANI-SUPP®), psyllium hydrophilic mucilloid (METAMUCIL®), sodium phosphate (FLEET ENEMA®), anti-tumor necrosis factor (TNF) (INFLIXIMAB, REMICADE®), or docusate (COLACE®, THEREVAC® PLUS).

For the treatment of sexual dysfunction related to multiple sclerosis, the second agent may be sildenafil (VIAGRA®), alprostadil (PROSTIN VR, MUSE), or papaverine.

For the treatment of spasticity, clonus, and/or muscle tics related to multiple sclerosis, the second agent may be diazepam (VALIUM®), clonazepam (KLONOPIN®, RIVOTRIL), baclofen (LIORESAL®), dantrolene sodium (DANTRIUM®), Tizanidine (ZANAFLEX®, SIRDALUD), clonidine (CATAPRES®), or botulinum toxin (BOTOX®, NERUOBLOC®).

For the treatment of tremors related to multiple sclerosis, the second agent may be clonazepam (KLONOPIN®, RIVOTRIL), gabapentin (NEUROTIN®), primidone (MYSOLINE®), botulinum toxin (BOTOX®, NEUROBLOC), actazolamide (DIAMOX®), and cabidopa-levodopa (SINEMET®), or isoniazid (LANIAZID, NYDRAZID®).

For the treatment of vertigo, nausea, and/or dizziness related to multiple sclerosis, the second agent may be meclizine (ANTIVERT®, BONAMINE), dienhydrinate (DRAMAMINE®), prochlorperazine (COMPAZINE®), scopolamine (TRANSDERM®), or diphenhydramine (BENADRYL®).

For the treatment of multiple sclerosis, the polypeptide mixture of the invention can be administered with or after therapy, such as, plasmaphoresis, reflexology, or total lymphoid radiation.

For the treatment of glaucoma or its symptoms, the second agent may be glatiramer acetate (COPAXONE®), pilocarpine (PILOCAR®, ISOPTO® CARPINE, PILOPINE HS®), isoptocarpine timolol hemihydrate (BETIMOL®), timolol maleate (BLOCADREN®, COSOPT®, TIMOLIDE®, TIMOPTIC®, TIMOPTIC-XE®), betaxolol (BETOPTIC®), levobunolol (BETAGAN®), carteolol (OCUPRESS®), metipranolol (OPTIPRANOLOL®), epinephrine (EPIPEN®, EPIFRIN®, EPPY/N®), dipivefrin (PROPINE®), carbachol (ISOPTO® CARBACHOL), apraclonidine (IOPIDINE®), brimonidine (ALPHAGAN®), dorzolamide (TRUSOPT®, COSOPT®), latanoprost (ZALATAN®), travaprost (TRAVATAN®), brimatoprost (LUMIGAN®), brinzolamide (AZOPT®) potent cholinesterase inhibitors (e.g. echothiophate iodide (PHOSPHOLINE IODIDE®), demecarium, isoblurophate, carbonic anhydrase inhibitors (e.g. dichlorphenamide (DARANIDE®) or acetazolamide), mannitol, oral glycerin, and mydriatics (e.g. homatropine, cyclopentolate, phenylephrine), memantine, or atropine.

For the treatment of glaucoma, the polypeptide mixture of the invention can be administered with or after therapy, such as, laser trabeculoplasty, filtering surgery, surgery, peripheral iridectomy, laser iridotomy, argon laser trabeculoplasty (ALT), selective laser trabeculoplasty (SLT), or neodymium (YAG laser cyclophotocoagulation).

For the treatment of inflammatory bowel disease (IBD) or its symptoms, the second agent may be glatiramer acetate (COPAXONE®), anticholinergics, diphenoxylate, loperamide, deodorized opium tincture, codeine, antibiotics, metronidazole (METROCREAM®, METROGEL®, METROGEL-VAGINAL®, METROLOTION®, METRO I.V.®, FLAGYL® I.V. RTU, FLAGYL® INJECTION, FLAGYL® ORAL, METRIC 21, PROTOSTAT, NORITATE®, and HELIDAC®), sulfasalazine (AZULFIDINE EN-TABS and ASULFIDINE), corticosteroid therapy (betamethasone (CELESTONE®, SOLUSPAN®), budesonide (ENTOCORT® EC), prednisone (DELTASONE®), methylprednisolone (MEDROL®, MEPROLONE UNIPAK, DEPO-MEDROL®, DEOJECT, DEPOPRED, DURALONE, M-PREDNISOL, MEDRALONE, SOLU-MEDROL®, DEMEDALONE) hydrocortisone (ANUSOL-HC®, CIPRO® HC OTIC, HYDROCORTONE®, COLOCORTM, CORTANE-B®, CORTEF®, CORTIC®-ND, LACTICARE®-HC, PROTOCORT®, PROCTOCREAM® HC, VYTONE®, ZOTO®-HC, ANUCORT-HC, ANUMED HC, CORT-DOME® HIGH POTENCY, HEMORRHOIDAL HC, HEMRIL-HC® UNISERTS, PROCTOCORT®), antimetabolites, immunosuppressive therapies (e.g., azathioprine (IMURAN®), 6-mercaptopurine (PURINETHOL®), cyclosporine (GENGRAFM, NEORAL®, SANDIMMUNE®), T lymphocyte aphaeresis, 4-amino quinolines, methotrexate (RHEUMATREX®, TREXALL®)), loperamide, 5-aminosalicylic acid (5-ASA) (mesalamine) (ASACOL®, PENTASA®, CLAVERSAL®, CANASA® SUPPOSITORY, ROWASA®), balsalazide (COLAZAL®), sulfasalazine (AZULFIDINE EN-TABS®), olsalazine (DIPENTUM®), azathioprine (AZASAN®, IMURAN®), ACTH 75, ACTH 120,®), anti-tumor necrosis factor (TNF) (INFLIXIMAB, REMICADE®), or antibiotics (e.g., ampicillin (PRINCIPEN), cefazolin).

For the treatment of IBD, the polypeptide mixture of the invention can be administered with or after therapy, such as, elemental diet, hyperalimentation, surgery, emergency colectomy, subtotal colectomy with ileostomy and rectosigmoid mucous fistula, or proctoclectomy with abdominoperineal resection.

For the treatment of Huntington's disease or its symptoms, the second agent may be glatiramer acetate (COPAXONE®), phenothiazine (chlorpromazine (THORAZINE®) 100 to 900 mg/day), butyrophenone neuroleptics (haloperidol), geldanamycin, RNA interference, trehalose, cystamine, rapamycin, glucocorticoids, nonsteroidal anti-inflammatory drugs (asprin, acetaminophen, ibuprofen (ADVIL®, MIDOL®)), omega-3 fatty acids (eicosapentaenoic acid (EPA) (LAX-101), docosahexanoic (DHA)), minocycline, folic acid, creatine, dichloroacetate, nicotinamide, riboflavin (BEVITAMEL® TABLETS, MEGA-B®, MASCOBAL® GEL, FOLGARD®, NIFREX®-150 FORTE CAPSULES, TRINSICON® CAPSULES), carnitine, tauroursodeoxycholic acid, ginkgo biloba, coenzyme Q10, vitamin A (MEGADOSE TABLETS, PALMITATE-A, VI-DAYLIN® ADC), vitamin C (PROFLAVANOL® 90 TABLETS, ACES® ANTIOXIDANT SOFT GELS, PERIDIN-CO TABLETS, TRINSICON® TABLETS, VI-DAYLIN® ADC), vitamin E (MEGADOSE TABLETS, UNIQUE E®, ACES® ANTIOXIDENT SOFT GELS, E-GEMSO SOFT GELS, LACTINOL-E® CREME), selenium (ACES® ANTIOXIDENT SOFT GELS), lipoic acid, arginine, mithramycin, remacemide, filuzole, lamotrigine (LAMICTAL®), memantine, gabapentin, HDAC inhibitors, retinoic acid or reserpine.

For the treatment of Amyotrophic Lateral Sclerosis (ALS)or its symptoms, the second agent may be riluzole (RILUTEK®), glatiramer acetate (COPAXONE®), baclofen, phenytoin (DILANTIN®), quinine, or amitriptyline.

For the treatment of ALS, the polypeptide mixture of the invention can be administered with or after therapy, such as, gastrostomy and noninvasive ventilation (e.g., BiPAP (bilevel positive airway pressure), or a tracheostomy and a ventilator).

Terms

The term “mixture” as used in this application in the phrase “mixture of polypeptides of the invention” means a mixture of copolymers of the amino acids comprising L-glutamic acid, L-alanine, L-tyrosine, and L-lysine, wherein some of the polypeptides of the mixture have C-terminal carboxyl groups and others have a diethylamide group. The polypeptides in the mixture do not all have the same amino acid sequence. Thus, each polypeptide in the mixture can vary from another in the order of amino acids and/or in the number of covalently bound amino acids. The polypeptide in the mixture may include residual impurities as a result of the manufacturing process. Because no reaction goes 100% to completion and, not all impurities can be totally eliminated, small amounts may remain and be present in the mixture. In general, said impurities are of the following three types:
Organic impurities, i.e. polypeptides containing protected amino acid residues such as 5-BZ-L-glutamyl and/or N6-TFA-L-Lysyl residues, originating from incomplete removal of the protecting groups during the production process. In addition, the polypeptide mixture of the invention molecules may contain brominated L-tyrosyl residues, formed during production due to the presence of free bromine in the HBr/acetic acid reagent.
The molecular structures of the identified organic impurities are related to the participating monomers, i.e. starting materials. They can also be quantified and referred to as follows:
   Residual trifluoroacetyl compounds (expressed as fluoride)
   Residual benzylated glutamyl residues (expressed as benzyl bromide)
   Residual brominated tyrosyl residues (expressed as bromotyrosine)
Unidentified organic impurities (determined by RP-HPLC): these are small molecular size polypeptides of the same origin with similar structures. These substances probably have similar response factors and the concentration (%) of each impurity can be calculated as % peak area relative to the polypeptide mixture of the invention peak area.
Residual solvents and inorganic impurities covered in the specification such as the residual solvent 1,4 dioxane, residual piperidine and heavy metals.

The term “average molecular weight” as used in this application means the molecular weight of the species of polypeptides present in the mixture in the highest relative proportion (i.e. the peak maximum) when the mixture is subjected to separation by molecular weight on an HPLC gel permeation column. This value can be obtained in several ways, e.g. from the retention time on a calibrated column; or from a correlation between the location of the peak and the location of the cochromatographed copolymer markers of defined sequence and molecular weight. Other methods of determining an average molecular weight such as by light scattering may be employed and will correspond substantially to the value obtained from the peak maximum.

The “average molecular weight” measured is that of the peptides in the mixture and, thus, is independent of the salt form of the peptides. Thus, a used herein, a mixture of polypeptides has the same average molecular weight irrespective of the polypeptides being in the form of the acetate salt or in the form of the tannate salt.

The term “carrier” refers to any binder, disintegrant, glidant, sweetening agent, flavoring, or any other vehicle with which the mixture is administered. Suitable carriers in the pharmaceutical composition may comprise a binder, such as microcrystalline cellulose, polyvinylpyrrolidone (polyvidone or povidone), gum tragacanth, gelatin, starch, lactose or lactose monochydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin; and/or a flavoring agent, such as peppermint, methyl salicylate, or orange flavoring.

The term “substantially” as used in this document means considerable in quantity or significantly great or being largely, but not wholly, that which is specified.

The term “substantially free” as used in this document means largely, but not wholly, chemically uncombined or not united with, attached to, combined with, or mixed with something specified.

The term “nanoparticle” as used in this document refers to a particle having an average diameter of 1-5000 nanometers (nm).

Experimental Details

Preparation of the mixture of polypeptides in the form of a tannate salt is readily accomplished by the conversion of the corresponding acetate salt of the polypeptides into the tannate salt as exemplified in the Experimental Details below. The tannate salt formation process described does not change the size or distribution of the peptides in the mixture.

### EXAMPLE 1A

### Synthesis of Glatiramer Tannate

A 5% aqueous solution containing 1.9 g of glatiramer acetate, with a pH of 5.5, was added to a 10% aqueous solution of tannic acid (USP grade) containing 2.95 g of tannic acid, with a pH of 3.2. The solutions were stirred for a few minutes and a suspension with a pH of 4.6 was formed. The suspension was centrifuged at 3220 G for 25 minutes and the colloidal decantate was separated. The solid sediment was washed with deionized water, and centrifuged again at 3220 G for 25 minutes. The clear decantate was discarded and the solid was dried under vacuum (25 mbar) at 40° C. and 2.6 g of dried solid was formed.

### EXAMPLE 1B

### Coagulation Using an Electrolyte

1 ml of NaCl aqueous solution with a density of 1.184 g/ml was added to 10 ml of the colloidal decantate from Example 1A, and then mixed. After 2 minutes, complete solid sedimentation was observed. The liquid layer was clear.

### EXAMPLE 1C

### Coagulation Using an Alcohol

1 ml of absolute ethanol was added to 10 ml of the colloidal decantate from Example 1A, and mixed. There was no immediate sedimentation. After 24 hours, no sedimentation had been observed.

### EXAMPLE 1D

### Coagulation Using an Polymer

1 ml of 4.8% aqueous starch solution was added to 10 ml of the colloidal decantate from Example 1A, and then mixed. After 15 minutes, slight sedimentation was observed. After 1 hour, significant sedimentation was observed. After 5 hours, the liquid was not entirely clear.

### EXAMPLE 2

A 5% aqueous solution containing 2.1 g of glatiramer in the form of glatiramer acetate, with a pH of 5.5, was added to a 10% aqueous solution of tannic acid (USP grade) containing 3.85 g of tannic acid, with a pH of 3.2. 5.2 g of NaCl was added, in the form of 10% aqueous solution, and the suspension was stirred for 30 minutes. The suspension was centrifuged at 3000 G for 22 minutes and the clear decantate was poured off.

The solid sediment was washed with 80 ml of deionized water followed by 2 ml of 10% NaCl solution, and centrifuged at 3000 G, and the clear decantate was poured off. The wet solid was dried under vacuum (25 mbar) at 40° C. 4.3 g of dried solid was attained, with a glatiramer content of 1.56 g (71% yield). The glatiramer content of the combined decantate was 0.64 g.

### EXAMPLE 3

A 5% aqueous solution containing 2.1 g of glatiramer in the form of glatiramer acetate with a pH of 5.5, was added to a 10% aqueous solution of tannic acid (USP grade) containing 6.0 g of tannic acid, with a pH of 3.2. 6.0 g of NaCl 10% aqueous solution was added, and the suspension was stirred for 2.25 hours. The suspension was filtered, and the solid cake was washed with aqueous 0.5% NaCl solution (31 g of solution). The wet solid was dried under vacuum (25 mbar) at 40° C. 6.2 g of dried solid was attained, with a glatiramer content of 1.9 g (86% yield).

The glatiramer content of the combined filtrate and wash was determined to be 0.3 g.

### EXAMPLE 4

A 5% aqueous solution containing 2.1 g of glatiramer in the form of glatiramer acetate, with a pH of 5.5, was added to a 10% aqueous solution of tannic acid (USP grade) containing 10.0 g of tannic acid, with a pH of 3.2. 9.1 g of 10% NaCl aqueous solution were added, and the suspension was stirred for 2.25 hours. The suspension was cooled to a temperature of 10° C. and filtered. The solid cake was washed with 80 g of 0.5% NaCl solution. The wet washed solid was dried under vacuum (25 mbar) at 40° C. 8.1 g of dry solid in the form of brownish pellets were attained. The amount of acetate ion in the sample was determined by gas chromatography to be 0.1%.

### EXAMPLE 5

### Reaction in Methanol and Isolation By Filtration

0.5 g Glatiramer acetate was dissolved in 20 ml methanol while stirred. A solution of 2.0 g of tannic acid in 10 ml methanol was added over 15 minutes. Precipitation of solids was observed during the addition. The suspension was stirred for one hour and settled. No solid sedimentation was observed. The solvent was distilled out under vacuum and the residue of evaporation (a brown semi-solid mass) was mixed with 20 ml methanol. The resulting suspension was stirred and filtered. Filtered solid was washed with methanol and dried under vacuum at 35° C. to constant weight, brownish powder material obtained.

The yield of dry solid was 0.77 g. An assay of Glatiramer by HPLC showed a yield of 41.4% by weight and a chemical yield of 76%.

### EXAMPLE 6

### Precipitation in Water in Presence of NaCl and Isolation by Decantation

1 g of Glatiramer acetate, 19 ml of deionized water and 1.8 ml of 10% NaCl were mixed. After stirring and complete dissolution of a solid solution of 4 g tannic acid in 36 ml deionized water was added. Precipitation of solid product took place during the addition. The suspension was stirred for 10 minutes and was allowed to settl for 5 minutes. Complete solid sedimentation was observed, liquor was decanted and 30 ml methanol was added. The product was suspended in methanol by stirring and solvent was distilled out under vacuum. The solid residue was dried at 35° C. under vacuum to a constant weight and ground in mortar “Substance A”. The decanted liquor was evaporated under vacuum to dryness then the solid residue was ground in mortar “Substance B”.

“Substance A”: Yield of dry solid—3.7 g, Assay of Glatiramer by HPLC 23.7% wt, Chemical yield 91%.

“Substance B”: Yield of dry solid—1.2 g, Assay of Glatiramer by HPLC 7.2% wt.

### EXAMPLE 7

### Precipitation in Water in Presence of NaCl and Isolation by Decantation

5 g Glatiramer acetate was dissolved in 95 ml deionized water. After stirring and complete dissolution, a solution containing of 20 g tannic acid and 1.8 ml of 10% NaCl in 180 ml deionized water was added. Precipitation of solid product took place during the addition. The suspension was stirred for 5 minutes and was allowed to settle for 5 minutes. Complete solid sedimentation was observed. The liquor was decanted and 100 ml methanol was added. The product was suspended in methanol by stirring and the solvent was distilled out under vacuum. The solid residue was dried at 35° C. under vacuum to a constant weight and ground in mortar “Substance A”. Decanted liquor was evaporated under vacuum to dryness then the solid residue was ground in a mortar “Substance B”.

“Substance A”: Yield of dry solid—18.4 g, Assay of Glatiramer by HPLC 23.5% wt, Chemical yield 90%.

“Substance B”: Yield of dry solid—8.0 g, Assay of Glatiramer by HPLC 5.4% wt.

### EXAMPLE 8

### Precipitation in Water in Presence of NaCl and Isolation by Decantation and Filtration in Ethanol

1 g Glatiramer acetate was dissolved in 19 ml deionized water. After stirring and complete dissolution, a solution containing 4 g tannic acid and 1.8 ml of 10% NaCl in 19 ml deionized water was added over 10 minutes. Precipitation of a solid product took place during the addition. The suspension was stirred for 5 minutes and was allowed to settle for one hour. Complete solid sedimentation was observed, liquor was decanted and 50 ml methanol was added. The product was suspended in ethanol, stirred for one hour and the resulting suspension of semi-solid material was filtered. The solid product was dried at 35° C. under vacuum to a constant weight and ground in mortar.

Yield of dry solid—1.2 g, Assay of Glatiramer by HPLC 49.3% wt, Chemical yield 70%.

### EXAMPLE 9

### Precipitation in Water in Presence of NaCl and Isolation by Decantation and Solvent Evaporation (Ethanol)

1 g Glatiramer acetate was dissolved in 19 ml deionized water. After stirring and complete dissolution, a solution of containing 4 g tannic acid and 1.8 ml of 10% NaCl in 19 ml deionized water was added over 10 minutes. Precipitation of solid product took place during the addition. The suspension was stirred for 5 minutes and was allowed to settle for one hour. Complete solid sedimentation was observed, liquor was decanted and 70 ml ethanol was added. The product was suspended in ethanol by stirring one then the solvent was distilled out under vacuum and stirring. An additional 30 ml of ethanol was introduced and evaporated to dryness under the same conditions. The solid residue was dried at 35° C. under vacuum to constant weight and ground in mortar.

### EXAMPLE 10

### Reaction in Hexane, Isolation by Filtration

0.5 g of Glatiramer acetate, 2 g of tannic acid and 35 ml hexane were mixed and stirred for 2 hours. The suspension was filtered (fast filtration), and the solid product was dried at 35° C. under vacuum to constant weight and ground in mortar.

Yield of dry solid—2.4 g, Assay of Glatiramer by HPLC 16.6% wt, Chemical yield 90%

### EXAMPLE 11

### Characterization of Glatiramer Tannate from Examples 2-4

The products of Examples 2-4 were analyzed using size exclusion HPLC to determine the glatiramer content of the glatiramer tannate product.

The yield is expressed in Table 1 in terms of percentage by weight of starting glatiramer recovered in the solid tannate.

TABLE 1
Glatiramer Content of Glatiramer Tannate Product

| | Ratio of | | |
|---|---|---|---|
| | tannic acid | Glatiramer | |
| | to | content in | |
| | glatiramer | product (% | Yield (% by |
| Example | (w/w) | by weight) | weight) |
| 2 | 1.75 | 36.3 | 71 |
| 3 | 2.72 | 30.6 | 86 |
| 4 | 4.54 | 26.9 | 94 |

Results

From Table 1, it can be noted that a higher ratio of tannic acid to glatiramer promotes greater glatiramer tannate yield by weight in the final product.

### EXAMPLE 12

### In vitro Testing of Glatiramer Tannate

The GT used in this example was prepared according to Example 4.

Simulated gastric fluid (SGF) with desired proteolytic activity was prepared based on USP procedure. 2.0 g of sodium chloride and 3.2 g of purified pepsin derived from porcine stomach mucosa, with an activity of 800 to 2500 units per mg of protein, was dissolved in 7.0 mL of hydrochloric acid and sufficient water to make 1000 mL. This test solution has a pH of about 1.2.

Simulated intestinal fluid (SIF) with desired proteolytic activity was prepared based on USP procedure. 6.8 g of monobasic potassium phosphate was dissolved in 250 mL of water by mixing. 77 mL of 0.2 N sodium hydroxide and 500 mL of water were added. 10.0 g of pancreatin were added, and the solution was mixed. The pH of the resulting solution was adjusted with either 0.2 N sodium hydroxide of 0.2 N hydrochloric acid to a pH of 6.8±0.1. The solution was diluted with water to 1000 mL.

Degradation in SGF

SGF was heated to 37.5±0.5° C. At time zero, Glatiramer acetate (GA) solution and Glatiramer tannate (GT) suspension were each added to separate samples of SGF to attain final concentration of about 1.5 mg/ml and 5 mg/ml respectively (molar equivalent amounts of glatiramer). The reactions were sampled at pre-determined intervals. When removing samples, the reaction was terminated in each sample using NaOH solution. The retention time (RT) of the glatiramer peak of each sample was analyzed using size exclusion chromatography. A higher glatiramer RT indicates reduction in molecular weight, which is indicative of degradation. The Relative Retention time (RRT) was calculated for each sample as (RT of Glatiramer/RT of acetone) and the results were shown in Table 2.

TABLE 2
Relative retention time of GA and GT in Simulated
Gastric Fluid (SGF)

| Time (min) | GA RRT | GT RRT |
|---|---|---|
| 0 | 0.69 | 0.73 |
| 2 | 0.75 | 0.78 |
| 5 | 0.76 | 0.80 |
| 10 | 0.77 | 0.80 |
| 20 | 0.78 | 0.81 |
| 30 | 0.78 | 0.81 |

Degradation in SIF

SIF was heated to 37.5±0.5° C. At time zero, Glatiramer acetate (GA) solution and Glatiramer tannate (GT) suspension were each added to separate samples of SIF to attain final concentration of about 1.5 mg/ml and 5 mg/ml respectively. The reactions were performed while continuously stirring. The reactions were sampled at chosen intervals. The reaction was terminated in each sample using concentrated HCl. The retention time (RT) of the glatiramer peak of each sample was analyzed using size exclusion chromatography. A higher glatiramer RT is indicative of greater degradation. The Relative Retention time (RRT) was calculated for each sample as (RT of Glatiramer/RT of acetone) and the results were shown in Table 3.

TABLE 3
Relative retention time of GA and GT in Simulated
Intestinal Fluid (SIF)

| Time (min) | GA RRT | GT RRT |
|---|---|---|
| 0 | 0.68 | 0.67 |
| 2 | No peak | 0.68 |
| 5 | No peak | 0.68 |
| 10 | No peak | 0.67 |
| 20 | No peak | 0.67 |
| 30 | No peak | 0.67 |

Results

In simulated gastric fluid, the degradations of GA and GT were similar. However, in simulated intestinal fluid, GA rapidly degrades. This degradation is evident from the absence of a glatiramer peak even after just two minutes of reaction. GT, though, does not degrade in SIF, even after 30 minutes of reaction.

### EXAMPLE 13

### Determination of Immunologic Activity of Glatiramer Tannate—Ex-vivo Testing of GT in CJSL Mice

Studies were conducted to test the potential properties of the mixture of polypeptides of the invention. In particular, the studies were to determine whether the mixture of polypeptides of the invention would elicit an immune response with similar cytokine patterns as those elicited by glatiramer acetate when tested in an ex-vivo model in mice.

Beginning on Day 0, groups of 4 CJSL Mice were fed daily with GA reference standard (GA RS, 12.5 μg/kg). Glatiramer Tannate (amount equivalent to 18.75 μg/kg), tannic acid (negative control, in an amount equivalent to the tannate administered to the 18.75 μg/kg glatiramer tannate group) or phosphate buffered saline (PBS—placebo control). During period of study, subsets of mice in each treatment group were sacrificed on days 4, 7 and 11. Spleens were excised and primary cell cultures were prepared. The effect of the treatment was tested by in-vitro activation of splenocytes with GA RS 50 μg/ml. The response of the cells to the challenge is a measure of previous exposure to Glatiramer and the generation of glatiramer specific T cells. T-cell response was monitored by detection of cytokines secreted from activated cells by ELISA analysis. The levels of IL-2, IL-3 and IL-5 cytokines were examined.

| | |
|---|---|
| BQL | Below Quantifiable Limit |
| BDL | Below Detectable Limit |
| AQL | Above Quantifiable Limit |
| PBS | Phosphate Buffer Saline |
| | (Placebo Group) |
| GA-RS | Glatiramer Acetate |
| | Reference Standard |

TABLE 4
Kinetics of IL-2, IL-3, IL-5 secretion by 50 μg/ml
GA RS in-vitro

| | | Glatiramer | Day | | | |
|---|---|---|---|---|---|---|
| | | Treatment | of | | | |
| | Formulation | dose | treat- | IL-2 | IL-3 | IL-5 |
| Group | administered | Mg/kg | ment | pg/ml | Pg/ml | Pg/ml |
| A1 | PBS | NA | 4 | BQL | BQL | BDL |
| A2 | PBS | NA | 7 | 24.8 | 102.7 | BDL |
| A3 | PBS | NA | 11 | 27.4 | 53.4 | BDL |
| B1 | GA-RS | 12.5 | 4 | BQL | BQL | BDL |
| B2 | GA-RS | 12.5 | 7 | 121.5 | 456.5 | BQL |
| B3 | GA-RS | 12.5 | 11 | 119.0 | 701.6 | BQL |
| C1 | Tannic Acid | NA | 4 | BQL | BQL | BDL |
| C2 | Tannic Acid | NA | 7 | 26.7 | 76.9 | BQL |
| C3 | Tannic Acid | NA | 11 | 16.6 | 63.2 | BQL |
| D1 | Glatiramer Tannate | 12.5 | 4 | BQL | BQL | BDL |
| D2 | Glatiramer Tannate | 12.5 | 7 | 31.8 | 215.3 | BQL |
| D3 | Glatiramer Tannate | 12.5 | 11 | 164.2 | 517.1 | 215 |
| E1 | Glatiramer Tannate | 18.75 | 4 | 32.7 | 16.0 | BQL |
| E2 | Glatiramer Tannate | 18.75 | 7 | 204.2 | AQL | BQL |
| E3 | Glatiramer Tannate | 18.75 | 11 | 149.7 | 841.2 | 97 |

Results

The results from Table 4 demonstrate unexpected results. Oral intake of the mixture of polypeptides of the invention over the course of 4, 7 and 11 days provided a similar immune response to that of GA-specific T cells, as evident by IL-2 secretion. The daily treatment of the CBJL mice with Glatiramer Tannate induced glatiramer specific spleen cells, secreting detectable levels of IL-2, IL-3 and IL-5 cytokines in response to in-vitro stimulation with GA. A dose response of the GT treatment was detected for the IL-2 and IL-3 secretion response. No significant difference was observed between the strength of the IL-2 secretion between the GA-RS and the polypeptides of the invention. Secretion of IL-2 and IL-3 increased with the GA-RS and with both concentrations of GT intake in the course of 7 days. Secretion of IL-2 and IL-3 increased following 11 days of treatment with the low dose of GT (12.5 mg/kg). However, secretion of both IL-2 and IL-3 decreased following 11 days of treatment with the high dose of GT (18.5 mg/kg). An unexpected negative dose response was observed for IL-5 secretion as well in the presence of the polypeptides of the invention.

The results indicate that the glatiramer of the glatiramer tannate composition is immunoavailable in mice following daily treatment. Furthermore, treatment with glatiramer tannate, has unexpectedly induced IL-5 (Th2) secretion.

### EXAMPLE 14

### Synthesis of the Tannate Salt of the Mixture of Polypeptides Disclosed in PCT International Application Publication No. WO 2006/029411

Tannic acid solution was prepared by dissolving 10.04 g of Tannic acid (Merck) in 95.0 g deionized water, to obtain about 100 ml of brown solution.

The mixture of polypeptides disclosed in PCT International Application Publication No. WO 2006/029411 in the form of an acetate solution was prepared by dissolving 2.5 g of the acetate salt in 47.5 g deionized water to obtain about 50 ml of solution.

The tannic acid solution was introduced into the mixture in solution over a period of 2 hours while stirring at ambient temperature. Solid precipitate was formed during the introduction and the pH of the mixture dropped from 5 to 3. The resulting milky suspension was stirred and 9.5 g 10% NaCl solution was added to the suspension.

Stirring was stopped and the suspension settled. The upper clear solution (122 ml) was decanted and the sediment (about 25 ml) was centrifuged at 1000 G for 10 min. The clear decantate was separated and 10.9 g of wet sediment was dried under vacuum at 35° C. to a constant mass. 7.7 g of dried solid (brown flakes) were ground in a mortar and a fine brownish powder of the mixture of polypeptides in the form of a tannate salt was obtained.

### EXAMPLE 15

### Synthesis of the Tannate Salt of the Mixture of Polypeptides Disclosed in PCT International Application Publication No. WO 2006/029411

Tannic acid solution was prepared by dissolving 6.7 g of Tannic acid (Merck) in 95.0 g deionized water, to obtain about 101 ml of brown solution.

The mixture of polypeptides from PCT International Application Publication No. WO 2006/029411 in the form of an acetate solution prepared by dissolving 2.5 g of the acetate salt, in 48.5 g of deionized water to obtain about 51 ml of solution.

The tannic acid solution was introduced into the mixture in acetate solution over 1 hour while stirring at ambient temperature. Solid precipitate was formed during the introduction and the mixture pH dropped from 5 to 3. The resulting milky suspension was stirred and 9.5 g 10% NaCl solution was added to the suspension.

Stirring was stopped and the suspension settled. The upper clear solution was decanted (127 ml) and the sediment (about 21 ml) was centrifuged at 1000 G for 10 min. The clear decantate was separated and 9.4 g of wet sediment was dried under vacuum at 35° C. to a constant mass. 6.9 g of dried solid (brown flakes) were ground in mortar and a fine brownish powder of the mixture of polypeptides in the form of a tannate salt was obtained.

Discussion

Unexpected Differences Between the Invention and the Prior Art

Example 7 shows that the biological effects of the tannate salt of the mixture of polypeptides of the invention are similar to those of GA. The invention, however, provides an unexpected improvement over the prior art. While the tannate salt exhibits biological reactivity similar to the acetate salt mixture, as shown in Table 3, the tannate salt exhibits a reduced propensity to degrade in simulated intestinal fluid (SIF). Over a period of thirty minutes, the tannate salt of the polypeptide mixture of the invention degraded slower in the SIF than GA, which immediately degraded. This indicates that the tannate salt of the mixture can be successfully formulated in oral dosage form for administration to humans.

In addition the tannate salt of the mixture of the invention has advantageous physical properties. Unlike GA, the tannate salt of the mixture is not hygroscopic. This property allows the tannate salt of the mixture to be easily milled or micronized and to be easily formulated into solid pharmaceutical forms.

The tannate salt of the mixture of the invention demonstrates a high melting point. Despite its amorphous nature, the tannate salt of the polypeptide mixture of the invention melts at a temperature of approximately 190° C.

In addition, tannate salt of the polypeptide mixture of the invention forms a gel upon the addition of water. This can be advantageous when formulating an oral form.

1. A composition comprising a mixture of polypeptides in the form of a tannate salt wherein each polypeptide consists of the amino acids L-glutamic acid, L-alanine, L-tyrosine and L-Lysine, and wherein the polypeptides in the mixture do not all have the same amino acid sequence.

2. The composition of claim 1, wherein the amino acids are present in the mixture in an amount such that the average molar fraction of amino acids is: L-glutamic acid 0.129-0.153; L-alanine 0.392-0.462; L-tyrosine 0.086-0.100; and L-lysine 0.300-0.374.

3. The composition of claim 2, wherein the amino acids are present in the mixture in an amount such that the average molar fraction of the amino acids is: L-glutamic acid 0.141; L-alanine 0.427; L-tyrosine 0.095; and L-lysine 0.338.

4. The composition of claim 1, wherein in the mixture the polypeptides have an average molecular weight from 2000 to 40,000 Daltons.

5-8. (canceled)

9. The composition of claim 4, wherein in the mixture the polypeptides have an average molecular weight from 4,700 to 11,000 Daltons.

10-26. (canceled)

27. The composition of claim 1, wherein the composition is lyophilized.

28. A pharmaceutical composition comprising a therapeutically effective amount of the composition of claim 1 and a pharmaceutically acceptable carrier.

29-40. (canceled)

40. A process for making a mixture of tannate salt of polypeptides, wherein each polypeptide consists of the amino acids L-glutamic acid, L-alanine, L-tyrosine and L-lysine, and wherein the polypeptides in the mixture do not all have the same amino acid sequence, comprising:
a) obtaining a mixture of acetate salt of polypeptides, wherein each polypeptide consists of the amino acids L-glutamic acid, L-alanine, L-tyrosine and L-lysine, and wherein the polypeptides in the mixture do not all have the same amino acid sequence; and
b) contacting the mixture of acetate salt of the polypeptides of step a) with tannic acid under suitable conditions to thereby form the mixture of tannate salt of the polypeptide.

41-44. (canceled)

45. A method of treating a human subject afflicted with an autoimmune disease comprising administering to the subject a therapeutically effective amount of the pharmaceutical composition claim 28, so as to treat the human subject.

46. A method of treating a human subject afflicted with an inflammatory non-autoimmune disease, an immune mediated disease, or a disease associated with demyelination comprising administering to the human subject a therapeutically effective amount of the pharmaceutical composition of claim 28, so as to treat the human subject.

47. A method of alleviating a symptom of an autoimmune disease in a subject afflicted with such a disease, comprising administering to the human subject the of the pharmaceutical composition of claim 28 in an amount effective to alleviate the symptom.

48. A method of alleviating a symptom of an inflammatory non-autoimmune disease, an immune mediated disease, or a disease associated with demyelination in a subject afflicted with such a disease, comprising administering to the human subject the pharmaceutical composition of claim 28 in an amount effective to alleviate the symptoms.

49. A method of promoting nerve regeneration or preventing or inhibiting secondary degeneration which may otherwise follow primary nervous system injury in a human subject comprising administering to the human subject a therapeutically effective amount of the pharmaceutical composition of claim 28.

50. A method of treating a human subject afflicted with a neurodegenerative disease comprising administering to the human subject a therapeutically effective amount of the pharmaceutical composition of claim 28 so as to thereby treat the human subject.

51. A method of alleviating a symptom of an neurodegenerative disease comprising administering to the human subject the pharmaceutical composition claim 28 in an amount effective to alleviate the symptom.

52-60. (canceled)

61. A method of treating a human subject afflicted with an inflammatory bowel disease comprising administering to the human subject a therapeutically effective amount of the pharmaceutical composition of claim 28 so as to treat of the inflammatory bowel disease.

62. A method of alleviating a symptom of an inflammatory bowel disease comprising administering to the human subject the pharmaceutical composition of claim 28 in an amount effective to alleviate the symptom.

63-67. (canceled)

68. A method of treating a human subject afflicted with multiple sclerosis comprising administering to the human subject a therapeutically effective amount of the pharmaceutical composition of claim 28 so as to thereby treat the human subject afflicted with multiple sclerosis.

69. A method of alleviating a symptom of multiple sclerosis in a human subject afflicted with multiple sclerosis comprising administering to the human subject the pharmaceutical composition of claim 28 in an amount effective to alleviate the symptom of multiple sclerosis.

70. A method of reducing the frequency of relapses in a human subject afflicted with relapse remitting multiple sclerosis comprising administering to the human subject a therapeutically effective amount of the pharmaceutical composition of claim 28 so as to thereby reduce the frequency of relapses in the human subject.

71. A method of reducing the disability based on the EDSS scale of a human subject afflicted with multiple sclerosis comprising administering to the human subject a therapeutically effective amount of the pharmaceutical composition of claim 28 so as to thereby reduce the disability based on EDSS scale in the human subject.

72. A method of reducing lesions detected by magnetic resonance imagining (MRI) in a human subject afflicted with multiple sclerosis comprising administering to the human subject a therapeutically effective amount of the pharmaceutical composition of claim 28 so as to thereby reduce the lesions detected by MRI in the human afflicted with multiple sclerosis.

73-86. (canceled)
